(19) United States
(12) Patent Application Publication
(10) Pub. No.: US 2020/0033307 A1
(43) Pub. Date: Jan. 30, 2020

(54) METHOD OF DETERMINING AN AMOUNT OF WATER IN A SAMPLE USING A DERIVATIVE OF IMIDAZOLE AND A HYDROGEN HALIDE DONOR

(71) Applicant: HONEYWELL INTERNATIONAL INC., Morris Plains, NJ (US)

(72) Inventor: Roman Neufeld, Hannover (DE)

(73) Assignee: HONEYWELL INTERNATIONAL INC., Morris Plains, NJ (US)

(21) Appl. No.: 16/520,706

(22) Filed: Jul. 24, 2019

Related U.S. Application Data
(60) Provisional application No. 62/703,118, filed on Jul. 25, 2018.

(51) Int. Cl. G01N 31/16; C07D 233/58

(52) U.S. Cl. CPC G01N 31/168; C07D 233/58

(57) ABSTRACT

A method for determining an amount of water in a sample includes utilizing a reagent and includes sulfur dioxide or derivative thereof, a protic or aprotic solvent or combinations thereof, a derivative of imidazole that has the following structure:

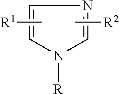

wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom, provided that R, R¹, and R² are not all hydrogen atoms. The reagent also includes a hydrogen halide donor. A molar ratio of the derivative of imidazole to the sulfur dioxide or derivative thereof is greater than 1:1. The method may include the step of providing a source of iodine.

### CROSS-REFERENCE TO RELATED APPLICATIONS

This application claims the benefit of priority to U.S. Provisional Patent Application Ser. No. 62/703,118, filed Jul. 25, 2018, the contents of which are herein incorporated by reference in their entirety.

### TECHNICAL FIELD

The present disclosure generally relates to a method of determining an amount of water in a sample using a derivative of imidazole and a hydrogen halide donor. The present disclosure more specifically relates to use of sulfur dioxide or a derivative thereof wherein a molar ratio of the derivative of imidazole to the sulfur dioxide or derivative thereof is greater than 1:1.

### BACKGROUND

The determination of water according to the Karl Fischer method, i.e., via Karl Fischer titration, utilizes the following reactions:

In an alcoholic or protic solution:

SO₂+ROH+B→BHSO₃R; (1)

BHSO₃R+I₂+H₂O+2B→BHSO₄R+2BHI; (2)

In a non-alcoholic or aprotic solution:

SO₂+I₂+H₂O+3B→BSO₃+2BHI; (3)

BSO₃+H₂O+B→BHSO₄+BH (4)

wherein B is a base and ROH is an alcohol. This titration is carried out in two basic forms, namely as a volumetric titration and as a coulometric titration.

In the classic Karl Fischer titration, a reagent includes an alkyl sulfite, which is oxidized to form an alkyl sulfate in the presence of water. Karl Fischer titrations are typically carried out in an alcoholic solution (such as methanol) or in the presence of the stoichiometric or a minimum amount of alcohol. However, the presence of the alcohol limits the applicability of such titrations because the alcohol can interfere with the titration and/or cause side reactions, thereby leading to inaccurate results.

For example, it is possible for acids, together with any alcohol of the reagents, to take part in an esterification reaction which results in the formation of water, thereby reducing the accuracy of the titration. Said differently, one of the primary disadvantages of using an alcohol is the likelihood of the alcohol undergoing water releasing side reactions with ketones, aldehydes, acetates and borates in the solution being titrated. When an amount of water is determined in ketones, ketal formation may occur, which similarly proceeds with the elimination of water, but which can be repressed if a halogenated alcohol such as chloroethanol and trifluoroethanol, which are toxic, is used as a solvent. Accordingly, it is difficult to measure the accurate water content of such compounds. The use of alcohols does have the advantage that alcohols stabilize the stoichiometry of the Karl Fischer reaction wherein the ratio of reacted iodine to water is 1:1.

The use of reagents which include SO₂ and pyridine has also been described wherein pyridine is used in excess. However, in such systems, the determinable water equivalent is heavily dependent on experimental conditions. For example, in such systems, a pyridine-SO₃ adduct forms, which takes part in a water-consuming side reaction (reaction (4)) that can falsify the titration results.

Fats and long-chained hydrocarbons are only sparingly soluble in alcohols, which had led to the use of halogenated hydrocarbons as a solvent component. However, their toxicity typically limits their use in such titrations. There are several other commercially available reagents that use alcohol derivatives (e.g. glycol ethers or toxic halogenated alcohols) in order to reduce the side reaction. However, significant sources of error still remain.

Moreover, a difficulty in using non-alcoholic (aprotic) Karl-Fischer reagents is the change in stoichiometry. Depending on the aprotic solvent and base used, the iodine to water ratio in the Karl Fischer reaction changes to 1:1-2 (instead of 1:1). If hydrolysis of the base-SO₃ adduct can be suppressed then the stoichiometry of I₂:H₂O remains 1:1.

Accordingly, there remains an opportunity to develop a Karl-Fischer reagent which allows highly accurate titrations in alcoholic and in non-alcoholic solvents.

### BRIEF SUMMARY

This disclosure provides a method for determining an amount of water in a sample. The method includes the step of providing a reagent and includes sulfur dioxide or a derivative thereof and a derivative of imidazole. The derivative of imidazole has the following structure:

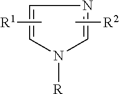

wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom, provided that R, R¹, and R² are not all hydrogen atoms. The reagent also includes a hydrogen halide donor. The reagent also includes a protic or aprotic solvent or combinations thereof. A molar ratio of the derivative of imidazole to the sulfur dioxide or derivative thereof is greater than 1:1. The method also includes the step of titrating the sample with the reagent. This method is typically described as a coulometric method and iodine is typically generated electronically.

This disclosure also provides a method for determining an amount of water in a sample wherein the method includes providing the aforementioned reagent, combining the sample with the reagent, and adding a source of iodine to the sample and/or the reagent. Typically, the reagent includes iodine. This method is typically described as a volumetric method. This disclosure also provides the aforementioned reagent itself.

### BRIEF DESCRIPTION OF THE DRAWINGS

The present disclosure will hereinafter be described in conjunction with the following drawing figures, wherein like numerals denote like elements, and

FIG. 1 is a graph showing low initial sample drift as set forth in the Examples relative to titration of a Li-battery electrolyte with 2% vinylene carbonate; and

FIG. 2 is a graph showing low initial sample drift as set forth in the Examples relative to titration of pure acetone.

### DETAILED DESCRIPTION

The following detailed description is merely exemplary in nature and is not intended to limit the method or reagent. Furthermore, there is no intention to be bound by any theory presented in the preceding background or the following detailed description.

Embodiments of the present disclosure are generally directed to methods of titration and solutions for the same. For the sake of brevity, conventional techniques may not be described in detail herein. Moreover, the various tasks and process steps described herein may be incorporated into a more comprehensive procedure or process having additional steps or functionality not described in detail herein. In particular, various steps in of titration are well-known and so, in the interest of brevity, many conventional steps will only be mentioned briefly herein or will be omitted entirely without providing the well-known process details. Various desirable features and characteristics of the present disclosure will become apparent from the subsequent detailed description of the disclosure and the appended claims, taken in conjunction with the accompanying drawings and the background of the disclosure.

This disclosure provides a method for determining an amount of water in a sample. Typically, this method can be described as a version or variant of the Karl Fisher Titration that is used to determine an amount of water or moisture in a sample. There are generally two methods used to perform the Karl Fischer titration. The first is known as a volumetric Karl Fischer titration. In this titration, the determination of the amount of water in the sample is based on an amount, or volume, of reagent used to convert the water. In this titration, samples are dissolved in a solvent before the titration begins. A reagent is added until the water is removed.

The second method is known as a coulometric Karl Fischer titration. In this titration, a reagent and solvent are combined in a titration cell. When a sample is introduced into the titration cell and dissolved, iodine is released by the induction of an electrical current. The amount of current required to convert the water is determinant of the amount of water in the sample. An advantage of the coulometric Karl Fischer titration is the capability to accurately measure small amounts of water, e.g. as low as 0.1 microgram (μg) of water. Each titration is described in greater detail below.

Referring now to the sample itself, the sample may be any sort of sample that includes water. The amount of water in the sample is not particularly limited and may be chosen by one of skill in the art. For example, in coulometric titrations, the amount of water in the sample is from about 0.1 to about 10000 μg of water, of from about 0.1 to about 3000 μg, of from about 20 to about 3000 μg of water, or of from about 20 to about 10000 μg of water. In volumetric titrations, the amount of water can greatly exceed 10000 μg. In still other embodiments, the maximum amount of water is determined by the size of the vessel used because of the amount of the reagent that would be required. The sample may be a liquid, gas, or solid provided that the sample includes an amount of water therein. The sample is typically a liquid that includes an amount of water therein. In various embodiments, the reagent of this disclosure is used with traditionally problematic samples that suffer from side reactions with traditional reagents such as solutions of ketones and/or aldehydes and unsaturated compounds such as vinylene carbonate.

The method includes the step of providing the reagent. The reagent may be alternatively described as a “Karl Fischer reagent.” The reagent is used in titrating the sample that includes the amount of water therein. For example, the reagent can be used in either Karl Fischer method described above, e.g. volumetric or coulometric titrations. The regent may be described as a titrating solution, e.g. when used in coulometric titrations. In volumetric titrations, e.g. in one or two component reagents, the reagent of this disclosure may act as a solvent. Additionally, the mixture of iodine and the reagent may act as a one-component reagent.

The reagent can be free of an alcohol or may include an alcohol. Typically, the terminology “free of” describes embodiments that include less than 30, 20, 10, 5, 4, 3, 2, 1, 0.5, 0.1, 0.05, or 0.01, weight percent of alcohol based on a total weight of the reagent. In one embodiment, the reagent is completely free of alcohol (i.e., includes zero weight percent or an amount of alcohol that falls below typical detection limits). Alternatively, the reagent may include any alcohol including, but not limited to, methanol, ethanol, propanol, mono- and di-ethylene glycol monoalkyl ethers, and combinations thereof.

The reagent includes sulfur dioxide or derivative thereof, a derivative of imidazole, a hydrogen halide donor, and a protic or aprotic solvent or combinations thereof. In one embodiment, the hydrogen halide donor is a hydrohalide of a second derivative of imidazole, e.g. a hydroiodide of the second derivative of imidazole. In one embodiment, the hydrogen halide donor is a hydrobromide of a second derivative of imidazole. In another embodiment, the hydrogen halide donor is a hydrochloride of a second derivative of imidazole. The reagent may be, consist essentially of, or consist of, the aforementioned compounds. The terminology “consisting essentially of” may describe embodiments that are free of compounds that are not hydrogen halide donors.

The terminology “consisting essentially of” may alternatively describe embodiments that include, or are free of, one or more soluble halides that aren't the aforementioned hydrogen halide donor or the hydrohalide of the second derivative of imidazole. For example, the reagent may include, or be free of, one or more of sodium halide, or halides of organic cations, such as tetrabutylammonium iodide, imidazole hydrogen iodide or trimethylamine hydrogen iodide and/or dissociating organic salts such as, for example, tetrabutylammonium chloride, diethanolamine hydrogen bromide, guanidinium salts such as guanidinium benzoate, and/or combinations thereof. The reagent may include, or be free of, imidazole itself. The reagent may also include, or be free of, nitrogen bases such as salts or carboxylic acids, such as tetramethylammonium acetate, trimethylammonium acetate, tetrabutylammonium benzoate, lithium propionate acetic acid, propionic acid, butyric acid, benzoic acid, buffer substances such diethanolammonium benzoate or imidazolium acetate, or combinations thereof.

Throughout this description, it is contemplated that whenever the word “halide” is used, any halide may be used, i.e., fluorine, chlorine, bromine, iodine, or combinations thereof, in various non-limiting embodiments. Moreover, in other non-limiting embodiments, any time the word “iodide” is used, this may be substituted for fluoride, chloride, or bromide.

Referring back, the reagent includes the derivative of imidazole and the sulfur dioxide (SO₂) or derivative thereof. The terminology “derivative thereof” describes compounds that act the same or substantially similarly to sulfur dioxide in the Karl-Fischer titration, as would be understood by one of skill in the art. For example, derivatives that may be used include, but are not limited to, reducing agents, sulfites such as dimethylsulfite, diethylsulfite, and combinations thereof.

The reagent includes a molar ratio of the derivative of imidazole to the sulfur dioxide or derivative thereof that is greater than 1:1. In other words, this disclosure does not utilize a 1:1 molar ratio of the derivative of imidazole to the sulfur dioxide or derivative thereof. In various embodiments, the molar ratio of the derivative of imidazole to the sulfur dioxide or derivative thereof is about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, about 15:1, about 15.5:1, about 16:1, about 16.5:1, about 17:1, about 17.5:1, about 18:1, about 18.5:1, about 19:1, about 19.5:1, or about 20:1. In various embodiments, if a liquid imidazole derivative is used, then the molar ratio can be much higher than 20:1, e.g. 30:1, 40:1, 50:1, or even higher. In one embodiment, the molar ratio of the derivative of imidazole to the sulfur dioxide or derivative thereof is greater than 2:1. In another embodiment, the molar ratio of the derivative of imidazole to the sulfur dioxide or derivative thereof is greater than 5:1. In a further embodiment, the molar ratio of the derivative of imidazole to the sulfur dioxide or derivative thereof is about 14:1. Moreover, it is contemplated that the reagent may include amounts “greater than” any of the aforementioned ratios, e.g., “greater than” about 2:1, greater than about 2.5:1, etc. In various non-limiting embodiments, it is also contemplated that all values and ranges of values, both whole and fractional, between and including those values set forth above are expressly contemplated for use herein.

In one additional embodiment, the sulfur dioxide or derivative thereof is present in an amount of from about 0.05 to about 5, mols/liter of the reagent. In other embodiments, the sulfur dioxide or derivative thereof is present in an amount of from about 0.05 to about 1, from about 0.1 to about 1, or from about 0.1 to about 0.5, mols/liter of the reagent. In various non-limiting embodiments, it is also contemplated that all values and ranges of values, both whole and fractional, between and including those values set forth above are expressly contemplated for use herein.

Referring now to the derivative of imidazole, this derivative may be described as a “first derivative” of imidazole, especially when a “second” derivative is used, as described below. It is to be understood that the “derivative of imidazole” and the “first derivative of imidazole” may be used interchangeably throughout.

The first derivative of imidazole has the following structure:

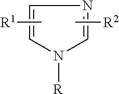

wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom. In this structure, R, R¹, and R² cannot all be hydrogen atoms because then the structure would be imidazole itself. In various embodiments, the first hydrocarbyl group has 1, 2, 3, 4, 5, or 6 carbon atoms. The second hydrocarbyl group may also independently include 1, 2, 3, 4, 5, or 6 carbon atoms wherein at one or more points in the chain of the group, a heteroatom including, but not limited to, nitrogen, oxygen, phosphorous, chlorine, bromine, or iodine. Moreover, each of R¹ and R² may be located at any point on the ring. In one additional embodiment, each of R, R¹, and R² is independently a hydrogen atom or a methyl, ethyl, propyl, or butyl group, provided that R, R¹, and R² are not all hydrogen atoms.

In various embodiments, the first derivative of imidazole is present in the reagent in the amounts set forth above relative to the sulfur dioxide or derivative thereof. In other embodiments, the first derivative of imidazole is present in an amount of from about 0.5 to about 5.5, or about 0.5 to about 5, or about 0.5 to about 2.5, mols/liter of the reagent. In other embodiments, the first derivative of imidazole is present in an amount that reflects one or more of the aforementioned molar ratios of the first derivative of imidazole to the sulfur dioxide or derivative thereof of greater than 1:1. For example, whatever the number of moles of the sulfur dioxide or derivative thereof is in the reagent, the first derivative of imidazole may be present in a number of moles that is greater than 1:1, e.g. in any of the ratios set forth above or when used in excess, e.g. as solvent. In various non-limiting embodiments, it is also contemplated that all values and ranges of values, both whole and fractional, between and including those values set forth above are expressly contemplated for use herein.

The reagent may also include a hydrogen halide acceptor having a pK_{A} of more than 5. This acceptor may be any known in the art including, but not limited to, an optionally substituted aliphatic, cyclic, heterocyclic or aromatic amines such as pyridine and derivatives thereof, trialkylamines, such as trimethylamine, triethylamine, tri-n-propylamine, tri-n-butylamine, N,N-dimethylethylamine, N,N-diethylmethylamine, N,N-dimethyl-n-butylamine, also N,N,N′,N′-tetramethylethylenediamine, imidazole, 1-methylpiperidine, 1-ethylpiperidine, 1,2-dimethylpyrrolidine, 2-(dimethylamino)-2-methyl-1-propanol, 1-methylpyrrolidine, N-ethylmorpholine, N-methylmorpholine, 2-morpholinoethanol, and combinations thereof. In various embodiments, the hydrogen halide acceptor is chosen from 2-morpholinoethanol, 2-(dimethylamino)-2-methyl-1-propanol, diethanol amine, and combinations thereof. In various embodiments, the acceptor is used in amount of from 0.005 to 5 mols/liter of the reagent. In various non-limiting embodiments, it is also contemplated that all values and ranges of values, both whole and fractional, between and including those values set forth above are expressly contemplated for use herein.

Referring now to the hydrogen halide donor, this donor may be a hydrogen halide of an amine. This amine may be any known in the art including imidazole such that the donor may be a hydrohalide of imidazole itself, e.g. a hydroiodide, hydrochloride, or hydrobromide. Moreover, this amine may be any described herein. In various embodiments, this amine is an optionally substituted aliphatic, cyclic, heterocyclic or aromatic amine such as pyridine and derivatives thereof, trialkylamine, such as trimethylamine, triethylamine, tri-n-butylamine, N,N-dimethylethylamine, N,N-diethylmethylamine, imidazole, N-ethylmorpholine, N-methylmorpholine, 2-morpholinoethanol, 1-methylpiperidine, 1-ethylpiperidine, 1-methylpyrrolidine, 2-(dimethylamino)-2-methyl-1-propanol, diethanol amine, pyridine and derivatives thereof, and combinations thereof. Accordingly, the hydrogen halide donor may be a hydrogen iodide/bromide/chloride of any of the above amines. The reagent may be free of any of the aforementioned hydrogen halide donors and instead utilize the hydrogen halide donor described immediately below.

In one embodiment, the hydrogen halide donor is a hydroiodide or hydrobromide or hydrochloride, or a combination thereof, of a second derivative of imidazole, wherein the second derivative of imidazole can have the following structure:

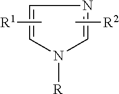

wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom. In one structure, R, R¹, and R² cannot all be hydrogen atoms. In another embodiment, each of R, R¹, and R² is a hydrogen atom. In various embodiments, the first hydrocarbyl group has 1, 2, 3, 4, 5, or 6 carbon atoms. The second hydrocarbyl group may also independently include 1, 2, 3, 4, 5, or 6 carbon atoms wherein at one or more points in the chain of the group, a heteroatom including, but not limited to, nitrogen, oxygen, phosphorous, chlorine, bromine, or iodine. Moreover, each of R¹ and R² may be located at any point on the ring. Each of R, R¹, and R² may be different from the aforementioned R, R¹, and R² of the first derivative of imidazole. Alternatively, each of R, R¹, and R² may be described as R³, R⁴, and R⁵, respectively. In one additional embodiment, each of R, R¹, and R² is independently a hydrogen atom or a methyl, ethyl, propyl, or butyl group, provided that R, R¹, and R² are not all hydrogen atoms.

The hydrohalide may be a hydroiodide, hydrochloride, or hydrobromide, or a combination thereof, of any of the aforementioned amines. In other words, the hydrohalide may be any compound of hydroiodic/bromic/chloric acid with any of the aforementioned amines or, e.g., any of the aforementioned embodiments of the second derivative of imidazole, as is appreciated by one of skill in the art. The second derivative of imidazole and the first derivative of imidazole may have the same general structure except that the second derivative of imidazole is a hydrohalide. In other words, the only difference between the first and second derivatives of imidazole may be that the first is not a hydrohalide and the second is a hydrohalide, even though the five membered ring structure and substituents may be the same or approximately the same.

The hydrogen halide donor may be any of the aforementioned compounds alone, may be the hydrohalide of the second derivative of imidazole alone, or may include a combination thereof.

The hydrogen halide donor may be present in any amount as chosen by one of skill in the art, e.g. in an amount of from about 0.01 to about 5, about 0.1 to about 2, about 0.2 to about 1.5, or from about 0.2 to about 1, mols/liter of the reagent. In various non-limiting embodiments, it is also contemplated that all values and ranges of values, both whole and fractional, between and including those values set forth above are expressly contemplated for use herein.

If the aprotic solvent is utilized, the aprotic solvent may be any known in the art including, but not limited to, ethers, such as diisopropyl ether, dibutyl ether, dioxane, tetrahydrofuran, diethylene glycol dimethyl ether, diethylene glycol diethylether, nitriles, such as acetonitrile, esters, such as ethyl acetate, ethyl propionate, isobutyl acetate, n-butyl acetate, ethylene carbonate, propylene carbonate, butyrolactone, halogenated hydrocarbons, such as chloroform, methylene chloride, carbon tetrachloride, bromoform, dibromomethane, 1,2-dichloropropane, acid amides, such as dimethylformamide, N-methylformamide, formamide, dimethylacetamide, 2-pyrrolidone, N-methylpyrrolidone, ketones, such as acetone, methyl ethyl ketone, diethyl ketone, cyclohexanone, methylcyclohexanone, ethylene carbonate, acetylacetone and other aprotic solvents, such as, for example, dimethylacetal. In one embodiment, the aprotic solvent is chosen from acetonitrile, propylene carbonate, ethyl acetate, tetrahydrofuran, dioxane, dimethylformamide or methylene chloride and combinations thereof. In a further embodiment, the aprotic solvent is chosen from cyclic and non-cyclic carbonates, ethers, esters, halo-hydrocarbons, acid amides, nitriles, ketones, glycol ethers, and combinations thereof. In another embodiment, the aprotic solvent is chosen from acetonitrile, ethylene carbonate, propylene carbonate, and combinations thereof. In another embodiment, the aprotic solvent is chosen from acetonitrile, propylene carbonate, and combinations thereof. In one embodiment, the aprotic solvent is acetonitrile. In another embodiment, the aprotic solvent is propylene carbonate. In another embodiment the aprotic solvent is dimethylformamide. In another embodiment aprotic solvent is chosen from dimethylformamide, acetonitrile and combinations thereof. In still other embodiments, the aprotic solvent may be pure (liquid) derivatives of imidazole, such as any described herein. It is contemplated that the reagent may be free of one or more of the aforementioned aprotic solvents or may include less than 5, 4, 3, 2, 1, 0.5, or 0.1, weight percent of one or more of the aforementioned aprotic solvents based on a total weight of the reagent. The aprotic solvent may be present in any amount as chosen by one of skill in the art and, for example, may be present in an amount to “balance” the aforementioned compounds so as to make the titrating composition have 100 total parts. In various non-limiting embodiments, it is also contemplated that all values and ranges of values, both whole and fractional, between and including those values set forth above are expressly contemplated for use herein.

If the protic solvent is used, the protic solvent may be any known in the art. For example, the protic solvent may be an alcohol such as methanol, ethanol, propanol, mono- and/or di-ethylene glycol monoalkylether having from 1, 2, 3, 4, 5, 6, 7, or 8 carbon atoms, or combinations thereof. The protic solvent may be used in any amount as described above relative to the aprotic solvent.

In one additional embodiment, the sulfur dioxide or derivative thereof is present in an amount of from about 0.05 to about 1, mols/liter of the reagent, the first derivative of imidazole is present in an amount of from about 0.5 to about 2.5 or about 0.5 to about 5, mols/liter of the reagent, the hydrohalide of the second derivative of imidazole is present in an amount of from about 0.01 to about 2, mols/liter of the reagent, and the solvent comprises a balance of the reagent. In another additional embodiment, the sulfur dioxide or derivative thereof is present in an amount of from about 0.10 to about 0.30, mols/liter of the reagent, the first derivative of imidazole is present in an amount of from about 0.5 to about 1, mols/liter of the reagent, the hydrohalide of the second derivative of imidazole is present in an amount of from about 0.1 to about 1.5, mols/liter of the reagent, and the solvent comprises a balance of the reagent. In still a further embodiment, the sulfur dioxide or derivative thereof is present in an amount of about 0.2 mols/liter of the reagent, the first derivative of imidazole is present in an amount of about 1.4, mols/liter of the reagent wherein each of R, R¹, and R² is independently a hydrogen atom or a methyl, ethyl, propyl, or butyl group, provided that R, R¹, and R² are not all hydrogen atoms, the hydrohalide of the second derivative of imidazole is present in an amount of about 0.2, mols/liter of the reagent, wherein each of R, R¹, and R² is independently a hydrogen atom or a methyl, ethyl, propyl, or butyl group, provided that R, R¹, and R² are not all hydrogen atoms; and the solvent comprises a balance of the reagent and is chosen from dimethylformamide, acetonitrile, propylene carbonate, trichloromethane and combinations thereof. In various non-limiting embodiments, it is also contemplated that all values and ranges of values between and including those values set forth above are expressly contemplated for use herein.

In one additional embodiment, e.g. as related to use of an alcoholic coulometric reagent, the sulfur dioxide or derivative thereof is present in an amount of from about 0.05 to about 1, mols/liter of the reagent, the first derivative of imidazole is present in an amount of from about 0.5 to about 2.5 or about 0.5 to about 5, mols/liter of the reagent, the hydrohalide of the second derivative of imidazole is present in an amount of from about 0.01 to about 2, mols/liter of the reagent, and the solvent comprises a balance of the reagent. In another additional embodiment, the sulfur dioxide or derivative thereof is present in an amount of from about 0.2 to about 1.0, mols/liter of the reagent, the first derivative of imidazole is present in an amount of from about 1.0 to about 1.7, mols/liter of the reagent, the hydrohalide of the second derivative of imidazole is present in an amount of from about 0.1 to about 1.1, mols/liter of the reagent, and the solvent comprises a balance of the reagent. In still a further embodiment, the sulfur dioxide or derivative thereof is present in an amount of about 0.9 mols/liter of the reagent, the first derivative of imidazole is present in an amount of about 1.2, mols/liter of the reagent wherein each of R, R¹, and R² is independently a hydrogen atom or a methyl, ethyl, propyl or butyl group, provided that R, R¹, and R² are not all hydrogen atoms, the hydrohalide of the second derivative of imidazole is present in an amount of about 0.9, mols/liter of the reagent, wherein each of R, R¹, and R² is independently a hydrogen atom or a methyl, ethyl, propyl or butyl group, provided that R, R¹, and R² are not all hydrogen atoms; and the protic solvent comprises a balance of the reagent and is chosen from methanol, ethanol, diethylenglycol monoethylether and combinations thereof. In various non-limiting embodiments, it is also contemplated that all values and ranges of values, both whole and fractional, between and including those values set forth above are expressly contemplated for use herein.

In another additional embodiment, e.g. as related to use of an alcoholic volumetric one-component reagent, the sulfur dioxide or derivative thereof is present in an amount of from about 0.01 to about 1, mols/liter of the reagent, the first derivative of imidazole is present in an amount of from about 0.5 to about 2.5 or about 0.5 to about 5, mols/liter of the reagent, the hydrohalide of the second derivative of imidazole is present in an amount of from about 0.01 to about 2, mols/liter wherein each of R, R¹, and R² is independently a hydrogen atom or a methyl, ethyl, propyl or butyl group, provided that R, R¹, and R² are not all hydrogen atoms, the hydrohalide of the second derivative of imidazole is present in an amount of about 0.3 to about 1.0, mols/liter of the reagent, wherein each of R, R¹, and R² is independently a hydrogen atom or a methyl, ethyl, propyl or butyl group, provided that R, R¹, and R² are not all hydrogen atoms; iodine is present in an amount of from about 0.5 to 5 mols/liter and the solvent comprises a balance of the reagent and is chosen from ethylene glycol monoalkylether comprises an alkyl group having from 1 to about 8 carbon atoms, diethylene glycol monoalkylether comprises an alkyl group having from 1 to about 8 carbon atoms and combinations thereof, and the solvent comprises a balance of the reagent. In various non-limiting embodiments, it is also contemplated that all values and ranges of values between and including those values set forth above are expressly contemplated for use herein.

Referring specifically to the step of providing the reagent, the reagent may be formed/provided using any order of addition. For example, any total amount or partial amount of any of the aforementioned components may be combined with any total amount or partial amount of any other of the components.

In one embodiment, for example, about 130 g of 1-methylimidazole is combined with about 130 g of 1-ethylimidazole hydrohalide and dissolved in about 250 mL of anhydrous propylene carbonate, about 250 mL of anhydrous ethylene carbonate and about 500 mL of anhydrous dimethyl formamide. Subsequently, about 8 g of sulfur dioxide or derivative thereof are introduced into the solution. In another embodiment, about 100 g of 1-methylimidazole and about 130 g of imidazole hydrohalide are dissolved in about 800 mL anhydrous propylene carbonate. About 6 g of sulfur dioxide or derivative thereof are then passed into the solution. In yet another example, these aforementioned reagents can be used in the anode space and/or the cathode space of the coulometric two chamber cell or as universal electrolyte in the single-chamber cell. These reagents of this disclosure can also be used as the solvent component of a one-component reagent or a two-component reagent. For example, if the reagent of this disclosure is used as a solvent, then a one-component reagent or a two-component reagent can be added thereto to titrate the water amount of a sample. If iodine is added to these reagents of this disclosure than the corresponding reagent can be used as a one-component reagent.

In one additional embodiment, for example, about 130 g of 1-methylimidazole is combined with about 130 g of 1-ethylimidazole hydroiodide and dissolved in about 250 mL of anhydrous propylene carbonate, about 250 mL of anhydrous ethylene carbonate and about 500 mL of anhydrous dimethyl formamide. Subsequently, about 8 g of sulfur dioxide or derivative thereof are introduced into the solution.

In another additional embodiment, 650 ml dimethyl formamide, 200 mL acetonitrile and 150 ml 1-ethylmidazole are mixed together. Subsequently, 25 g of SO₂ and then 28 g iodine are added to the solution. The dark brown solution is decolorized to bright orange color by adding dropwise water to the solution. In this process 1-ethylimidazole hydroiodide is produced inside the solution as hydrogen halide donor.

In still another additional embodiment, 140 ml 1-methylmidazole as hydrogen halide acceptor and 50 g 1-ethylimidazole hydroiodide as hydrogen halide donor are dissolved in a mixture of 700 ml dimethyl formamide and 150 mL acetonitrile. Subsequently, 20 g of SO₂ are introduced to the solution. The solution is dehydrated by adding about 7 g iodine.

In another embodiment, 100 g 2-morpholinoethanol and 100 g 2-ethylimidazole are dissolved in 800 ml methanol. Then 15 g of SO₂ are introduced into the solution while cooling. 8 g of iodine are added. The formation of 2-morpholinoethanol hydroiodide and 2-ethylimidazole hydroiodide as hydrogen halide donor inside the solution is managed by adding dropwise water to the brown solution till it turns to a pale-yellow color.

In a further embodiment, 60 g 2-(dimethylamino)-2-methyl-1-propanol and 100 g 2-ethylimidazole as hydrogen halide acceptor are dissolved in 840 ml methanol. 70 g 1-methylimidazole hydroiodide as hydrohalide donor are added to the solution. Then 15 g of SO₂ are introduced into the solution while cooling. The solution is dehydrated by adding iodine.

Solutions from the aforementioned examples can either be used as anolyte in a coulometric cell having only one chamber or additionally as catholyte in a coulometric cell having two separated chambers. Furthermore, solutions the aforementioned examples can also be filled into a volumetric titration cell as solvent component. The water containing sample can be added into a titration cell and titrated by using a commercially available iodine reagent (e.g. one-component or two-component reagent).

In one embodiment, the method includes the step of titrating the sample with the reagent. This is typically described as a volumetric method. In another embodiment, the method includes the step of combining the sample and the reagent such that the sample can be titrated. In this embodiment, the method typically includes the step of providing a source of iodine (I₂). The source of iodine may be any known in the art, e.g. solid I₂ dissolved in any suitable solvent and/or in any of the aforementioned reagents. In various embodiments, the solution to which the iodine is added may have from about 1 to about 10 weight percent of iodine after its addition. In a coulometric method, the iodine can be generated by anodic oxidation of an iodide such that no additional or external source of iodine may be needed/used. The sample can be titrated to determine the amount of water in the sample by using one of the aforementioned Karl Fischer methods. In various non-limiting embodiments, it is also contemplated that all values and ranges of values, both whole and fractional, between and including those values set forth above are expressly contemplated for use herein.

For example, any necessary iodine can be added via the aforementioned iodine solution or can be generated by anodic oxidation from added iodide. During method, the added or anodically generated iodine is typically reduced to iodide by the reaction with the sulfur dioxide or derivative thereof and water. When there is no more water, free iodine is left over. The iodine excess can be used for indicating the end-point, for example for visual or for photometric indication. It is also possible to indicate the end-point electrochemically, for example bipotentiometrically or biamperometrically.

Volumetric determination can be carried out by introducing the reagent into the titration vessel as a solvent component. Then the sample can be added to the titration vessel such that the water is titrated by introducing an iodine containing one-component reagent or a two-component reagent. Typically, titrations utilizing a one-component reagent that is traditionally a solution of iodine, base and SO₂ include providing a solvent in a vessel, adding the sample to the vessel that includes the solvent, and then adding the one-component reagent to the combination of the sample in the vessel and the solvent. The reagent of this disclosure can be used in this titration as a solvent. Titrations utilizing a two-component reagent typically include providing, e.g. a base and SO₂ containing solvent like the reagent of this disclosure in a vessel. Then a sample is typically added to the vessel. Finally, the two-component reagent is then typically added to the vessel such that the titration reactions can begin.

The reagent of this disclosure can also be used as a one-component titrating reagent in a volumetric titration. To use it as such it is necessary to add 1-10 weight % of iodine to the reagent.

Coulometric determination can be carried out, for example, by introducing the components of the reagent into a coulometric cell, such as a divided cell and then, according to the cell construction, adding the sample and electrolyzing, by switching on the electrolysis current, until the water present in the sample has been converted.

Prior to the determination of an amount of water in the sample, water contained in the aprotic solvent can be removed in a blank titration (e.g. by pre-electrolysis in the case of a coulometric determination). Typically, in a coulometric titration, the aforementioned first derivative of imidazole is combined with the hydrohalide of the second derivative of imidazole. In various embodiments, e.g. if the coulometric cell requires a reagent having a conductivity of from about 5 to about 20 mS/cm, it may be necessary to add additional supporting electrolytes. These may be soluble inorganic salts such as tetrabutylammonium chloride, imidazolium hydrogen bromide, etc.

To indicate the end-point, both in volumetric analysis and in coulometric titration, it is contemplated that bipotentiometric or biamperometric indication may be utilized. For example, the reagent and/or sample may be spiked with one or more known compounds that have known reproducible end-points. These may be chosen by those of skill in the art. Moreover, one or more buffers may be utilized. In still other embodiments, the method may include or be free of one or more of the compounds, method steps, etc. as set forth in U.S. Pat. No. 5,401,662.

In other embodiments, the hydrohalide of the second imidazole derivative and/or the hydrogen halide donor may be introduced to the reagent by (1) adding the second imidazole derivative or an amine used to form the hydrogen halide donor to the reagent along with a halogen acid, e.g. hydriodic acid, (utilizing an in-situ reaction) and/or (2) adding the hydrohalide of the second imidazole derivative and/or the hydrogen halide donor as an adduct that was prepared outside the reagent solution, i.e., in a separate reaction that is not considered in-situ.

In various embodiments, the method of this disclosure produces start drifts of less than about 15, 14, 13, 12, 11, 10, 9, 8, 7, 6, 5, 4, 3, 2, or 1, μg/min, as determined by one of skill in the art using any of the aforementioned titrating methods. In various non-limiting embodiments, it is also contemplated that all values and ranges of values, both whole and fractional, between and including those values set forth above are expressly contemplated for use herein.

In additional embodiments of this disclosure, it is contemplated that the reagent may be free of the hydrogen halide donor. In other embodiments, it is contemplated that the reagent may be as follows wherein all numbers are in approximate amounts of moles/liter of reagent.

| | | | Volumetric |
|---|---|---|---|
| | | | Alcohol Free or |
| | | | Alcoholic |
| | Coulometric | Coulometric | (One Component |
| Component | Alcohol Free | Alcoholic | Reagent) |
| Sulfur Dioxide or | 0.01 to 5 | 0.01 to 5 | 0.01 to 5 |
| Derivative Thereof | Or | Or | Or |
| | 0.2 to 0.3 | 0.6 to 0.9 | 0.4 to 0.9 |
| Derivative of Imidazole | 0.1 to 10 | 0.1 to 10 | 0.1 to 10 |
| | Or | Or | Or |
| | 1 to 1.8 | 1 to 1.8 | 1.5 to 3 |
| Hydrogen Halide | 0.01 to 3 | 0.01 to 3 | 0 to 3 |
| Donor | Or | Or | Or |
| | 0.15 to 0.3 | 0.3 to 0.9 | 0 to 1 |
| Hydrogen Halide | 0.0 to 10 | 0.0 to 10 | 0.0 to 10 |
| Acceptor | Or | Or | Or |
| | 0 to 1 | 0 to 2 | 0 to 2 |
| Free Iodine | — | — | 0.1 to 2 |
| | | | Or |
| | | | 0.1 to 1 |

In various non-limiting embodiments, it is also contemplated that all values and ranges of values, both whole and fractional, between and including those values set forth above are expressly contemplated for use herein.

### Examples

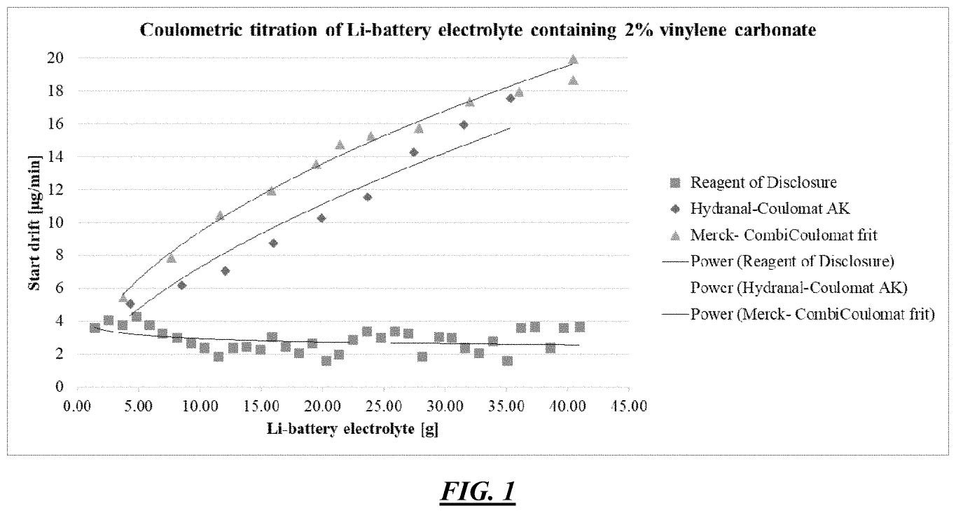

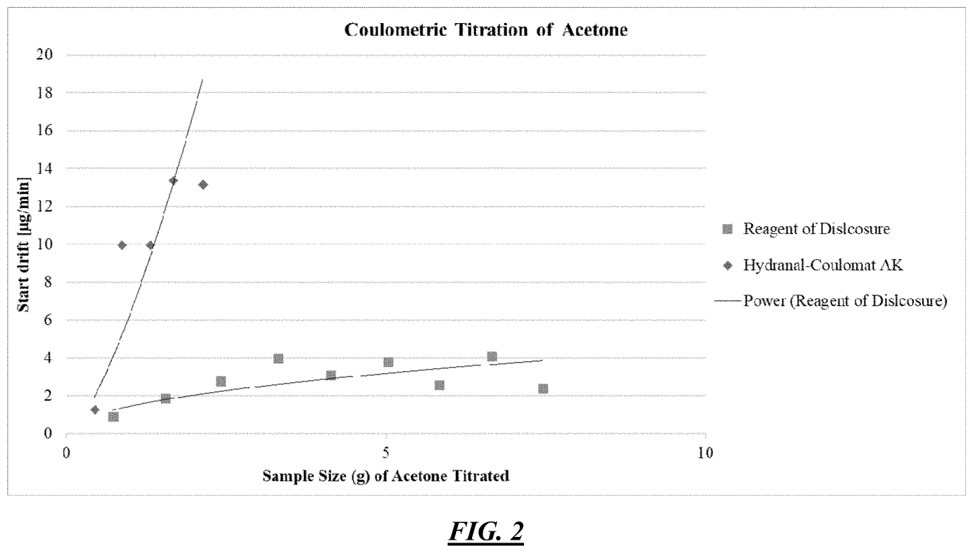

A series of titrations are performed according to this disclosure and as comparative examples. Results are set forth below in Tables 1-5 and in FIGS. 1 and 2.

A first set of examples involves titration of a Li-battery electrolyte with 2% vinylene carbonate using a Metrohm 852 Titrando apparatus. The global lithium-ion battery market grows very fast and the demand of highly accurate water determinations in lithium electrolytes increases day to day. It is important to measure the water content in lithium electrolytes since even low water quantities and other impurities may cause the battery to malfunction. Almost all modern Li-electrolytes include vinylene carbonate (VC). VC is typically necessary to stabilize the battery and increases its life expectancy. With current reagents, the water determination is limited to a few measurements, due to side reactions with VC that disturb the measurement and increase the error.

In the methodology of the Karl-Fischer titration it is very important to have a stable and low start drift. For very accurate results, the start drift should be below 10 μg/min. Typically, titrating devices will not start the titration if the start drift is above 20 μg/min. If commercially available reagents like Hydranal-Coulomat AK or Merck-CombiCoulomat frit are used for the titration of vinylene carbonate containing electrolytes, the starting drift increases after each injection as shown in the data below and in FIG. 1. The reason for that is a side reaction of the reagent with vinylene carbonate. The data set forth below and in FIG. 1 also shows that no/minimal side reaction occurs with the reagent of this disclosure as demonstrated by the stable and low initial drift. This corresponds to production of highly accurate results.

Examples (Ex.) 1-36 represent various embodiments of this disclosure wherein PC is propylene carbonate, CH₃CN is acetonitrile, 1-EtIMI is 1-ethylimidazole, 1-EtIMI-HI is a hydrohalide of 1-ethylimidazole, and SO₂ is sulfur dioxide. The results are set forth in Table 1. Examples (Ex.) 37-45 represent comparative examples wherein a first commercially available reagent is used (i.e., Hydranal-Coulomat AK). The results are set forth in Table 2. Examples (Ex.) 38-56 represent comparative examples wherein a second commercially available reagent is used (i.e., Merck-CombiCoulomat frit). The results are set forth in Table 3. The data from Tables 1-3 is summarized and set forth visually in FIG. 1 along with lines showing approximate averages.

The data set forth below and in FIG. 1 shows that the Examples 1-36 that represent this disclosure produce extremely small start drifts as compared to the comparative examples. This is due to the minimal side reactions that occur. These results show that the reagent of this disclosure can be used with traditionally difficult-to-titrate solutions that typically suffer from side-reactions thereby potentially ruining or even stopping titration altogether. In Table 1, “LiTFSI” is Lithium bis(trifluoromethanesulfonyl)imide.

TABLE 1

| | Reagent of | Sample | Water [ppm] | Sample Size | Water (μg) | Start Drift | Total Sample |
|---|---|---|---|---|---|---|---|
| Ex. | This Disclosure | Titrated | in Sample | [g] Titrated | in Sample | [μg/min] | Size (g) |
| 1 | PC + CH3CN | Sample 1 | 52.5 | 1.34 | 70.5 | 3.6 | 1.34 |
| | 1-EtIMI + | 1M | | | | | |
| | 1-EtIMI- | LiTFSI in | | | | | |
| | HI + SO2 | PC + 2% | | | | | |
| | | VC | | | | | |
| 2 | PC + CH3CN | Sample 1 | 56.8 | 1.16 | 65.8 | 4.1 | 2.50 |
| | 1-EtIMI +30 | 1M | | | | | |
| | 1-EtIMI- | LiTFSI in | | | | | |
| | HI + SO2 | PC + 2% | | | | | |
| | | VC | | | | | |
| 3 | PC + CH3CN | Sample 1 | 55.4 | 1.11 | 61.5 | 3.8 | 3.61 |
| | 1-EtIMI + | 1M | | | | | |
| | 1-EtIMI- | LiTFSI in | | | | | |
| | HI + SO2 | PC + 2% | | | | | |
| | | VC | | | | | |
| 4 | PC + CH3CN | Sample 1 | 55.2 | 1.15 | 63.7 | 4.3 | 4.77 |
| | 1-EtIMI + | 1M | | | | | |
| | 1-EtIMI- | LiTFSI in | | | | | |
| | HI + SO2 | PC + 2% | | | | | |
| | | VC | | | | | |
| 5 | PC + CH3CN | Sample 1 | 53 | 1.05 | 55.4 | 3.8 | 5.81 |
| | 1-EtIMI + | 1M | | | | | |
| | 1-EtIMI- | LiTFSI in | | | | | |
| | HI + SO2 | PC + 2% | | | | | |
| | | VC | | | | | |
| 6 | PC + CH3CN | Sample 2 | 47.8 | 1.06 | 50.9 | 3.3 | 6.88 |
| | 1-EtIMI + | 1M | | | | | |
| | 1-EtIMI- | LiTFSI in | | | | | |
| | HI + SO2 | PC + 2% | | | | | |
| | | VC | | | | | |
| 7 | PC + CH3CN | Sample 2 | 46.4 | 1.21 | 56.1 | 3 | 8.09 |
| | 1-EtIMI + | 1M | | | | | |
| | 1-EtIMI- | LiTFSI in | | | | | |
| | HI + SO2 | PC + 2% | | | | | |
| | | VC | | | | | |
| 8 | PC + CH3CN | Sample 2 | 46 | 1.13 | 52 | 2.7 | 9.22 |
| | 1-EtIMI + | 1M | | | | | |
| | 1-EtIMI- | LiTFSI in | | | | | |
| | HI + SO2 | PC + 2% | | | | | |
| | | VC | | | | | |
| 9 | PC + CH3CN | Sample 2 | 46.6 | 1.07 | 49.9 | 2.4 | 10.29 |
| | 1-EtIMI + | 1M | | | | | |
| | 1-EtIMI- | LiTFSI in | | | | | |
| | HI + SO2 | PC + 2% | | | | | |
| | | VC | | | | | |
| 10 | PC + CH3CN | Sample 2 | 47 | 1.19 | 55.9 | 1.9 | 11.48 |
| | 1-EtIMI + | 1M | | | | | |
| | 1-EtIMI- | LiTFSI in | | | | | |
| | HI + SO2 | PC + 2% | | | | | |
| | | VC | | | | | |
| 11 | PC + CH3CN | Sample 2 | 46.6 | 1.18 | 54.8 | 2.4 | 12.65 |
| | 1-EtIMI + | 1M | | | | | |
| | 1-EtIMI- | LiTFSI in | | | | | |
| | HI + SO2 | PC + 2% | | | | | |
| | | VC | | | | | |
| 12 | PC + CH3CN | Sample 2 | 46.3 | 1.05 | 48.6 | 2.5 | 13.70 |
| | 1-EtIMI + | 1M | | | | | |
| | 1-EtIMI- | LiTFSI in | | | | | |
| | HI + SO2 | PC + 2% | | | | | |
| | | VC | | | | | |
| 13 | PC + CH3CN | Sample 2 | 48.3 | 1.13 | 54.8 | 2.3 | 14.84 |
| | 1-EtIMI + | 1M | | | | | |
| | 1-EtIMI- | LiTFSI in | | | | | |
| | HI + SO2 | PC + 2% | | | | | |
| | | VC | | | | | |
| 14 | PC + CH3CN | Sample 2 | 48 | 0.98 | 47.1 | 3.1 | 15.82 |
| | 1-EtIMI + | 1M | | | | | |
| | 1-EtIMI- | LiTFSI in | | | | | |
| | HI + SO2 | PC + 2% | | | | | |
| | | VC | | | | | |
| 15 | PC + CH3CN | Sample 2 | 46.8 | 1.12 | 52.2 | 2.5 | 16.93 |
| | 1-EtIMI + | 1M | | | | | |
| | 1-EtIMI- | LiTFSI in | | | | | |
| | HI + SO2 | PC + 2% | | | | | |
| | | VC | | | | | |
| 16 | PC + CH3CN | Sample 2 | 46.7 | 1.12 | 52.1 | 2.1 | 18.05 |
| | 1-EtIMI + | 1M | | | | | |
| | 1-EtIMI- | LiTFSI in | | | | | |
| | HI + SO2 | PC + 2% | | | | | |
| | | VC | | | | | |
| 17 | PC + CH3CN | Sample 2 | 45.9 | 1.06 | 48.7 | 2.7 | 19.11 |
| | 1-EtIMI + | 1M | | | | | |
| | 1-EtIMI- | LiTFSI in | | | | | |
| | HI + SO2 | PC + 2% | | | | | |
| | | VC | | | | | |
| 18 | PC + CH3CN | Sample 3 | 59.3 | 1.11 | 65.9 | 1.6 | 20.22 |
| | 1-EtIMI + | 1M | | | | | |
| | 1-EtIMI- | LiTFSI in | | | | | |
| | HI + SO2 | PC + 2% | | | | | |
| | | VC | | | | | |
| 19 | PC + CH3CN | Sample 3 | 58.1 | 1.04 | 60.7 | 2 | 21.27 |
| | 1-EtIMI + | 1M | | | | | |
| | 1-EtIMI- | LiTFSI in | | | | | |
| | HI + SO2 | PC + 2% | | | | | |
| | | VC | | | | | |
| 20 | PC + CH3CN | Sample 3 | 57.8 | 1.16 | 67.2 | 2.9 | 22.43 |
| | 1-EtIMI + | 1M | | | | | |
| | 1-EtIMI- | LiTFSI in | | | | | |
| | HI + SO2 | PC + 2% | | | | | |
| | | VC | | | | | |
| 21 | PC + CH3CN | Sample 3 | 56.5 | 1.17 | 65.9 | 3.4 | 23.60 |
| | 1-EtIMI + | 1M | | | | | |
| | 1-EtIMI- | LiTFSI in | | | | | |
| | HI + SO2 | PC + 2% | | | | | |
| | | VC | | | | | |
| 22 | PC + CH3CN | Sample 3 | 56.5 | 1.10 | 62.1 | 3 | 24.70 |
| | 1-EtIMI + | 1M | | | | | |
| | 1-EtIMI- | LiTFSI in | | | | | |
| | HI + SO2 | PC + 2% | | | | | |
| | | VC | | | | | |
| 23 | PC + CH3CN | Sample 3 | 55.6 | 1.12 | 62.1 | 3.4 | 25.81 |
| | 1-EtIMI + | 1M | | | | | |
| | 1-EtIMI- | LiTFSI in | | | | | |
| | HI + SO2 | PC + 2% | | | | | |
| | | VC | | | | | |
| 24 | PC + CH3CN | Sample 3 | 57 | 1.15 | 65.3 | 3.3 | 26.96 |
| | 1-EtIMI + | 1M | | | | | |
| | 1-EtIMI- | LiTFSI in | | | | | |
| | HI + SO2 | PC + 2% | | | | | |
| | | VC | | | | | |
| 25 | PC + CH3CN | Sample 4 | 73.2 | 1.14 | 83.6 | 1.9 | 28.10 |
| | 1-EtIMI + | 1M | | | | | |
| | 1-EtIMI- | LiTFSI in | | | | | |
| | HI + SO2 | PC + 2% | | | | | |
| | | VC | | | | | |
| 26 | PC + CH3CN | Sample 4 | 71.9 | 1.33 | 95.5 | 3.1 | 29.43 |
| | 1-EtIMI + | 1M | | | | | |
| | 1-EtIMI- | LiTFSI in | | | | | |
| | HI + SO2 | PC + 2% | | | | | |
| | | VC | | | | | |
| 27 | PC + CH3CN | Sample 4 | 73.3 | 1.08 | 79.1 | 3 | 30.51 |
| | 1-EtIMI + | 1M | | | | | |
| | 1-EtIMI- | LiTFSI in | | | | | |
| | HI + SO2 | PC + 2% | | | | | |
| | | VC | | | | | |
| 28 | PC + CH3CN | Sample 4 | 72.6 | 1.09 | 79.5 | 2.4 | 31.61 |
| | 1-EtIMI + | 1M | | | | | |
| | 1-EtIMI- | LiTFSI in | | | | | |
| | HI + SO2 | PC + 2% | | | | | |
| | | VC | | | | | |
| 29 | PC + CH3CN | Sample 4 | 73.3 | 1.13 | 82.6 | 2.1 | 32.73 |
| | 1-EtIMI + | 1M | | | | | |
| | 1-EtIMI- | LiTFSI in | | | | | |
| | HI + SO2 | PC + 2% | | | | | |
| | | VC | | | | | |
| 30 | PC + CH3CN | Sample 4 | 72.9 | 1.14 | 82.9 | 2.8 | 33.87 |
| | 1-EtIMI + | 1M | | | | | |
| | 1-EtIMI- | LiTFSI in | | | | | |
| | HI + SO2 | PC + 2% | | | | | |
| | | VC | | | | | |
| 31 | PC + CH3CN | Sample 4 | 74.7 | 1.13 | 84.7 | 1.6 | 35.00 |
| | 1-EtIMI + | 1M | | | | | |
| | 1-EtIMI- | LiTFSI in | | | | | |
| | HI + SO2 | PC + 2% | | | | | |
| | | VC | | | | | |
| 32 | PC + CH3CN | Sample 4 | 72.2 | 1.16 | 83.5 | 3.6 | 36.16 |
| | 1-EtIMI + | 1M | | | | | |
| | 1-EtIMI- | LiTFSI in | | | | | |
| | HI + SO2 | PC + 2% | | | | | |
| | | VC | | | | | |
| 33 | PC + CH3CN | Sample 4 | 73.9 | 1.14 | 84.1 | 3.7 | 37.30 |
| | 1-EtIMI + | 1M | | | | | |
| | 1-EtIMI- | LiTFSI in | | | | | |
| | HI + SO2 | PC + 2% | | | | | |
| | | VC | | | | | |
| 34 | PC + CH3CN | Sample 5 | 78.6 | 1.22 | 95.7 | 2.4 | 38.52 |
| | 1-EtIMI + | 1M | | | | | |
| | 1-EtIMI- | LiTFSI in | | | | | |
| | HI + SO2 | PC + 2% | | | | | |
| | | VC | | | | | |
| 35 | PC + CH3CN | Sample 5 | 76.6 | 1.15 | 87.9 | 3.6 | 39.67 |
| | 1-EtIMI + | 1M | | | | | |
| | 1-EtIMI- | LiTFSI in | | | | | |
| | HI + SO2 | PC + 2% | | | | | |
| | | VC | | | | | |
| 36 | PC + CH3CN | Sample 5 | 78.7 | 1.30 | 102 | 3.7 | 40.96 |
| | 1-EtIMI + | 1M | | | | | |
| | 1-EtIMI- | LiTFSI in | | | | | |
| | HI + SO2 | PC + 2% | | | | | |
| | | VC | | | | | |

The data set forth below and also in FIG. 1 shows that the Examples 37-45 that represent comparative examples produce much larger start drifts as compared to the examples 1-36. This is due to the plentiful side reactions that occur. These results show that common reagents cannot typically be used with traditionally difficult-to-titrate solutions that typically suffer from side-reactions thereby likely ruining or even stopping titration altogether. In Table 2, “LP572” is commercially available from BASF and includes LiPF₆ as a conducting salt with 2% vinylene carbonate (VC).

TABLE 2

| | Comparative | Sample | Water [ppm] | Sample Size | Water (μg) | Start Drift | Total Sample |
|---|---|---|---|---|---|---|---|
| Ex. | Reagent | Titrated | in Sample | [g] Titrated | in Sample | [μg/min] | Size (g) |
| 37 | 100 mL | Sample 1 | 3.4 | 4.27 | 14.7 | 5.1 | 4.27 |
| | Coulomat | Electrolyte | | | | | |
| | AK // Coul | LP572 | | | | | |
| | CG-K | with | | | | | |
| | | 2% VC | | | | | |
| 38 | 100 mL | Sample 1 | 3.5 | 4.17 | 14.4 | 6.2 | 8.45 |
| | Coulomat | Electrolyte | | | | | |
| | AK // Coul | LP572 | | | | | |
| | CG-K | with | | | | | |
| | | 2% VC | | | | | |
| 39 | 100 mL | Sample 1 | 4.4 | 3.55 | 15.6 | 7.1 | 12.00 |
| | Coulomat | Electrolyte | | | | | |
| | AK // Coul | LP572 | | | | | |
| | CG-K | with | | | | | |
| | | 2% VC | | | | | |
| 40 | 100 mL | Sample 1 | 3.4 | 3.95 | 13.5 | 8.8 | 15.94 |
| | Coulomat | Electrolyte | | | | | |
| | AK // Coul | LP572 | | | | | |
| | CG-K | with | | | | | |
| | | 2% VC | | | | | |
| 41 | 100 mL | Sample 1 | 3.2 | 3.90 | 12.4 | 10.3 | 19.84 |
| | Coulomat | Electrolyte | | | | | |
| | AK // Coul | LP572 | | | | | |
| | CG-K | with | | | | | |
| | | 2% VC | | | | | |
| 42 | 100 mL | Sample 1 | 3.4 | 3.79 | 12.8 | 11.6 | 23.64 |
| | Coulomat | Electrolyte | | | | | |
| | AK // Coul | LP572 | | | | | |
| | CG-K | with | | | | | |
| | | 2% VC | | | | | |
| 43 | 100 mL | Sample 2 | 4.9 | 3.73 | 18.2 | 14.3 | 27.36 |
| | Coulomat | Electrolyte | | | | | |
| | AK // Coul | LP572 | | | | | |
| | CG-K | with | | | | | |
| | | 2% VC | | | | | |
| 44 | 100 mL | Sample 2 | 4.8 | 4.13 | 19.6 | 16 | 31.50 |
| | Coulomat | Electrolyte | | | | | |
| | AK // Coul | LP572 | | | | | |
| | CG-K | with | | | | | |
| | | 2% VC | | | | | |
| 45 | 100 mL | Sample 2 | 4.7 | 3.82 | 18.1 | 17.6 | 35.32 |
| | Coulomat | Electrolyte | | | | | |
| | AK // Coul | LP572 | | | | | |
| | CG-K | with | | | | | |
| | | 2% VC | | | | | |

The data set forth below and also in FIG. 1 shows that the Examples 46-56 that represent comparative examples produce much larger start drifts as compared to the examples 1-36. This is due to the plentiful side reactions that occur. These results show that common reagents cannot typically be used with traditionally difficult-to-titrate solutions that typically suffer from side-reactions thereby likely ruining or even stopping titration altogether. In Table 3, “LP572” is commercially available from BASF and includes LiPF₆ as a conducting salt with 2% vinylene carbonate (VC).

TABLE 3

| | Comparative | Sample | Water [ppm] | Sample Size | Water (μg) | Start Drift | Total Sample |
|---|---|---|---|---|---|---|---|
| Ex. | Reagent | Titrated | in Sample | [g] Titrated | in Sample | [μg/min] | Size (g) |
| 46 | 100 mL | Sample 1 | 27.6 | 3.65 | 100.7 | 5.5 | 3.65 |
| | Combi | Electrolyte | | | | | |
| | Coulomat | LP572 | | | | | |
| | frit // CG-K | with | | | | | |
| | | 2% VC | | | | | |
| 47 | 100 mL | Sample 1 | 27.3 | 3.96 | 108.2 | 7.9 | 7.60 |
| | Combi | Electrolyte | | | | | |
| | Coulomat | LP572 | | | | | |
| | frit // CG-K | with | | | | | |
| | | 2% VC | | | | | |
| 48 | 100 mL | Sample 1 | 27.4 | 3.96 | 108.3 | 10.5 | 11.56 |
| | Combi | Electrolyte | | | | | |
| | Coulomat | LP572 | | | | | |
| | frit // CG-K | with | | | | | |
| | | 2% VC | | | | | |
| 49 | 100 mL | Sample 1 | 27.5 | 4.19 | 115.2 | 12 | 15.75 |
| | Combi | Electrolyte | | | | | |
| | Coulomat | LP572 | | | | | |
| | frit // CG-K | with | | | | | |
| | | 2% VC | | | | | |
| 50 | 100 mL | Sample 1 | 27.7 | 3.66 | 101.3 | 13.6 | 19.41 |
| | Combi | Electrolyte | | | | | |
| | Coulomat | LP572 | | | | | |
| | frit // CG-K | with | | | | | |
| | | 2% VC | | | | | |
| 51 | 100 mL | Sample 1 | 28.3 | 1.95 | 55.2 | 14.8 | 21.36 |
| | Combi | Electrolyte | | | | | |
| | Coulomat | LP572 | | | | | |
| | frit // CG-K | with | | | | | |
| | | 2% VC | | | | | |
| 52 | 100 mL | Sample 1 | 28.2 | 2.52 | 71 | 15.3 | 23.88 |
| | Combi | Electrolyte | | | | | |
| | Coulomat | LP572 | | | | | |
| | frit // CG-K | with | | | | | |
| | | 2% VC | | | | | |
| 53 | 100 mL | Sample 2 | 31.4 | 3.96 | 124.4 | 15.8 | 27.84 |
| | Combi | Electrolyte | | | | | |
| | Coulomat | LP572 | | | | | |
| | frit // CG-K | with | | | | | |
| | | 2% VC | | | | | |
| 54 | 100 mL | Sample 2 | 31.3 | 4.12 | 128.8 | 17.4 | 31.96 |
| | Combi | Electrolyte | | | | | |
| | Coulomat | LP572 | | | | | |
| | frit // CG-K | with | | | | | |
| | | 2% VC | | | | | |
| 55 | 100 mL | Sample 2 | 31.8 | 4.02 | 127.9 | 18 | 35.98 |
| | Combi | Electrolyte | | | | | |
| | Coulomat | LP572 | | | | | |
| | frit // CG-K | with | | | | | |
| | | 2% VC | | | | | |
| 56 | 100 mL | Sample 2 | 47.8 | 4.44 | 212.2 | 18.7 | 40.42 |
| | Combi | Electrolyte | | | | | |
| | Coulomat | LP572 | | | | | |
| | frit // CG-K | with | | | | | |
| | | 2% VC | | | | | |

A second set of examples involves titration of pure acetone. Both aldehydes and ketones like acetone traditionally pose problems with Karl Fischer titrations because they form acetals and ketals, respectively, with conventional reagents. These reactions form water, which is also titrated, resulting in vanishing end points, erroneously high water content and increasing start drifts.

As described above, in the methodology of the Karl-Fischer titration it is very important to have a stable and low start drift. For very accurate results, the start drift should be below 10 μg/min. Typically, titrating devices will not start the titration if the start drift is above 20 μg/min. If commercially available reagents like Hydranal-Coulomat AK are used for the titration of acetone, the starting drift increases after each injection as shown in the data below and in FIG. 2. The data set forth below and in FIG. 2 also shows that no/minimal side reaction occurs with the reagent of this disclosure as demonstrated by the stable and low initial drift. This corresponds to production of highly accurate results.

The side reaction with ketones can be suppressed by replacing methanol in the titrating agent with another solvent. For example commercially available Hydranal-Coulomat AK is methanol-free. With a methanolic reagent, no titration of acetone would be possible. Although use of Hydranal-Coulomat AK that contains a less reactive alcohol allows for a few measurements to be performed, the start drift reaches the 20 μg/min mark after a view mL of acetone. The reagent of this disclosure allows for highly accurate titrations with fewer to no problems.

Examples (Ex.) 57-65 represent various embodiments of this disclosure. The results are set forth in Table 4. These titrations are performed using a Mettler Toledo C30S apparatus.

Examples (Ex.) 66-70 represent comparative examples wherein a first commercially available reagent is used (i.e., Hydranal-Coulomat AK). The results are set forth in Table 5. These titrations are performed using a Metrohm 852 Titrando apparatus.

The data from Tables 4 and 5 is summarized and set forth visually in FIG. 2 along with lines showing approximate averages.

The data shows that the Examples 57-65 that represent this disclosure produce extremely small start drifts as compared to the comparative examples. This is due to the minimal side reactions that occur. These results show that the reagent of this disclosure can be used with traditionally difficult-to-titrate samples that typically suffer from side-reactions thereby potentially ruining or even stopping titration altogether. In Table 4, the terminology “PC+1,2 DimethylIMI+IMI-HI+SO2” refers to propylene carbonate+1,2 dimethylimidazole+imidazole hydrohalide+SO₂.

TABLE 4

| | Reagent of | Acetone | Sample | Absolute | Start |
|---|---|---|---|---|---|
| | This | Titrated | Size [g] | water | Drift |
| Ex. | Disclosure | (g) | Titrated | present (μg) | [μg/min] |
| 57 | PC + 1,2 | 0.72037 | 0.72037 | 191.7 | 0.9 |
| | DimethylIMI + | | | | |
| | IMI-HI + | | | | |
| | SO2 | | | | |
| 58 | PC + 1,2 | 0.82158 | 1.54195 | 217.9 | 1.9 |
| | DimethylIMI + | | | | |
| | IMI-HI + | | | | |
| | SO2 | | | | |
| 59 | PC + 1,2 | 0.86592 | 2.40787 | 231.2 | 2.8 |
| | DimethylIMI + | | | | |
| | IMI-HI + | | | | |
| | SO2 | | | | |
| 60 | PC + 1,2 | 0.89617 | 3.30404 | 238.4 | 4 |
| | DimethylIMI + | | | | |
| | IMI-HI + | | | | |
| | SO2 | | | | |
| 61 | PC + 1,2 | 0.82172 | 4.12576 | 219 | 3.1 |
| | DimethylIMI + | | | | |
| | IMI-HI + | | | | |
| | SO2 | | | | |
| 62 | PC + 1,2 | 0.9057 | 5.03146 | 241.5 | 3.8 |
| | DimethylIMI + | | | | |
| | IMI-HI + | | | | |
| | SO2 | | | | |
| 63 | PC + 1,2 | 0.80055 | 5.83201 | 215.4 | 2.6 |
| | DimethylIMI + | | | | |
| | IMI-HI + | | | | |
| | SO2 | | | | |
| 64 | PC + 1,2 | 0.81225 | 6.64426 | 220.3 | 4.1 |
| | DimethylIMI + | | | | |
| | IMI-HI + | | | | |
| | SO2 | | | | |
| 65 | PC + 1,2 | 0.80248 | 7.44674 | 216.2 | 2.4 |
| | DimethylIMI + | | | | |
| | IMI-HI + | | | | |
| | SO2 | | | | |

The data set forth below and also in FIG. 2 shows that the Examples 66-70 that represent comparative examples produce much larger start drifts as compared to the examples 57-65. This is due to the plentiful side reactions that occur. These results show that common reagents cannot typically be used with traditionally difficult-to-titrate solutions that typically suffer from side-reactions thereby likely ruining or even stopping titration altogether.

TABLE 5

| | | | | Absolute | |
|---|---|---|---|---|---|
| | | Acetone | Sample | water | |
| | Comparative | Titrated | Size [g] | present | Start Drift |
| Ex. | Reagent | (ml) | Titrated | (μg) | [μg/min] |
| 66 | Hydranal- | 0.5 ml | 0.43313 | 102 | 1.3 |
| | Coulomat AK | | | | |
| 67 | Hydranal- | 0.5 ml | 0.864 | 132.3 | 10 |
| | Coulomat AK | | | | |
| 68 | Hydranal- | 0.5 ml | 1.30539 | 150.2 | 10 |
| | Coulomat AK | | | | |
| 69 | Hydranal- | 0.5 ml | 1.66761 | 128.9 | 13.4 |
| | Coulomat AK | | | | |
| 70 | Hydranal- | 0.5 ml | 2.13269 | 176.8 | 13.2 |
| | Coulomat AK | | | | |

The reagent described above and set forth in the aforementioned Examples provides various advantages. For example, the reagent operates using the same stoichiometric reaction as in commercially available KF reagents (the ratio H₂O:I₂ is 1:1). The reagent uses a higher stoichiometric base:SO2 ratio thereby accelerating the relevant reactions such that the formed base-SO₃ complex is stable. The reagent ensures highly accurate results for low and high water quantities. The reagent allows for almost unlimited quantities of acetone to be titrated accurately with a small increase of start drift. Typically, only a few grams of acetone can be titrated because of high increase in start drift and therefore a significant increase of error. The reagent can be used as an anolyte as well as a catholyte for coulometric titrations. The reagent can also be used as solvent component for a one-component reagent and/or a two-component reagent. If iodine is added to the reagent, then the reagent can also be used as a one-component titrating solution. In various embodiments, the reagent has an increased stability towards unsaturated hydrocarbons like vinylene carbonate that is used as an additive in many Li-battery-electrolytes. Moreover, almost all of the aforementioned compounds are typically non-toxic and non-CMR (Carcinogenic, Mutagenic and toxic to Reproduction).

In various non-limiting embodiments, it is contemplated that any terminology of alcohol-free solvent, solution, and/or reagent may be substituted with aprotic solvent, solution, and/or reagent. Similarly, in various non-limiting embodiments, it is contemplated that any terminology of alcoholic solvent, solution, and/or reagent may be substituted with protic solvent, solution, and/or reagent.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A method for determining an amount of water in a sample, said method comprising the steps of:
A. providing a reagent comprising:
(1) sulfur dioxide or a derivative thereof;
(2) a derivative of imidazole having the following structure: 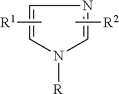
wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom, provided that R, R¹, and R² are not all hydrogen atoms;
(3) a hydrogen halide donor; and
(4) a protic or aprotic solvent or combinations thereof,
wherein a molar ratio of the derivative of imidazole to the sulfur dioxide or derivative thereof is greater than 1:1; and
B. titrating the sample with the reagent.

2. The method of claim 1 wherein the hydrogen halide donor is a hydrohalide of a second derivative of imidazole wherein the second derivative of imidazole has the following structure: 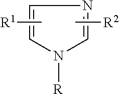
wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom.

3. The method of claim 2 wherein:
the sulfur dioxide or derivative thereof is present in an amount of from about 0.01 to about 5, mols/liter of the reagent,
the derivative of imidazole is present in an amount of from about 0.1 to about 10, mols/liter of the reagent,
the hydrohalide of the second derivative of imidazole is present in an amount of from about 0.01 to about 3, mols/liter of the reagent,
wherein the reagent optionally comprises a hydrohalide acceptor present in an amount of from 0 to about 10 mols/liter of the reagent, and
wherein the solvent comprises a balance of the reagent.

4. The method of claim 2 wherein:
the sulfur dioxide or derivative thereof is present in an amount of from about 0.2 to about 0.9, mols/liter of the reagent,
the derivative of imidazole is present in an amount of from about 1 to about 1.8, mols/liter of the reagent,
the hydrohalide of the second derivative of imidazole is present in an amount of from about 0.15 to about 0.9, mols/liter of the reagent,
wherein the reagent optionally comprises a hydrohalide acceptor present in an amount of from 0 to about 2 mols/liter of the reagent, and
wherein the solvent comprises a balance of the reagent.

5. The method of claim 1 wherein the derivative of imidazole has the following structure: 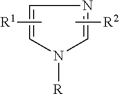
wherein each of R, R¹, and R² is independently a hydrogen atom or a methyl, ethyl, propyl, or butyl group, provided that R, R¹, and R² are not all hydrogen atoms.

6. The method of claim 2 wherein the second derivative of imidazole has the following structure: 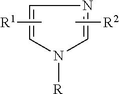
wherein each of R, R¹, and R² is independently a hydrogen atom or a methyl, ethyl, propyl, or butyl group, provided that R, R¹, and R² are not all hydrogen atoms.

7. The method of claim 1 wherein the solvent is an aprotic solvent and is chosen from propylene carbonate, acetonitrile, N,N-dimethylformamide, N-methylformamide, acetamide formamide, N-methyl acetamide, N-dimethyl acetamide, 2-pyrrolidone, N-methyl-2-pyrrolidone, chloroform, dichloromethane, bromoform, dibromomethane, and/or the said solvent is an protic solvent and comprises a balance of the reagent and chosen from methanol, ethanol, propanol, diethylene glycol monoethyl ether, 1-methoxy-2-propanol and combinations thereof.

8. The method of claim 1 wherein the molar ratio of the derivative of imidazole to the sulfur dioxide or derivative thereof is greater than 2:1.

9. The method of claim 1 wherein the reagent further comprises a hydrogen halide acceptor optionally chosen from 2-morpholinoethanol, 2-(dimethylamino)-2-methyl-1-propanol, diethanol amine, and combinations thereof.

10. A method for determining an amount of water in a sample, said method comprising the steps of:
A. providing a reagent and comprises:
(1) sulfur dioxide or a derivative thereof,
(2) a derivative of imidazole having the following structure: 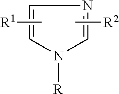
wherein each of R, R¹, and R² is independently a hydrogen atom or a methyl, ethyl, propyl, or butyl group, provided that R, R¹, and R² are not all hydrogen atoms.
(3) a hydrogen halide donor; and
(4) a protic or aprotic solvent or combinations thereof;
wherein a molar ratio of the derivative of imidazole to the sulfur dioxide or derivative thereof is greater than 1:1;
B. combining the sample with the reagent; and
C. adding a source of iodine to the sample and/or the reagent.

11. The method of claim 11 wherein the hydrogen halide donor is a hydrohalide of a second derivative of imidazole wherein the second derivative of imidazole has the following structure: 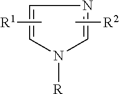
wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom.

12. The method of claim 10 wherein the reagent further comprises a hydrogen halide acceptor optionally chosen from 2-morpholinoethanol, 2-(dimethylamino)-2-methyl-1-propanol, diethanol amine, and combinations thereof.

13. The method of claim 10 wherein:
the sulfur dioxide or derivative thereof is present in an amount of from about 0.01 to about 5, mols/liter of the reagent,
the derivative of imidazole is present in an amount of from about 0.1 to about 10, mols/liter of the reagent,
the hydrohalide of the second derivative of imidazole is present in an amount of from about 0.01 to about 3, mols/liter of the reagent,
wherein the reagent further comprises iodine present in an amount of from about 0.1 to about 2 mols/liter of the reagent, and
wherein the solvent comprises a balance of the reagent.

14. A reagent comprising:
(1) sulfur dioxide or derivative thereof;
(2) a derivative of imidazole having the following structure: 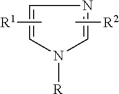
wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom, provided that R, R¹, and R² are not all hydrogen atoms;
(3) a hydrogen halide donor; and
(4) a protic or aprotic solvent or combinations thereof,
wherein a molar ratio of said derivative of imidazole to said sulfur dioxide or derivative thereof is greater than 1:1.

15. The reagent of claim 14 wherein said hydrogen halide donor is a hydrohalide of a second derivative of imidazole wherein the second derivative of imidazole has the following structure: 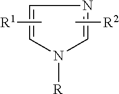
wherein each of R, R¹, and R² is independently a hydrogen atom, a phenyl group, a substituted phenyl group, a first hydrocarbyl group having from 1 to 6 carbon atoms, or a second hydrocarbyl group having 1 to 6 carbon atoms interrupted in at least one position with a heteroatom.

16. The reagent of claim 15 wherein the sulfur dioxide or derivative thereof is present in an amount of from about 0.01 to about 5, mols/liter of the reagent,
the derivative of imidazole is present in an amount of from about 0.1 to about 10, mols/liter of the reagent,
the hydrohalide of the second derivative of imidazole is present in an amount of from about 0.01 to about 3, mols/liter of the reagent,
wherein the reagent optionally comprises a hydrohalide acceptor present in an amount of from 0 to about 10 mols/liter of the reagent, and
wherein the solvent comprises a balance of the reagent.

17. The reagent of claim 14 wherein said derivative of imidazole has the following structure: 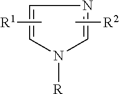
wherein each of R, R¹, and R² is independently a hydrogen atom or a methyl, ethyl, propyl, or butyl group, provided that R, R¹, and R² are not all hydrogen atoms;

18. The reagent of claim 14 wherein said second derivative of imidazole has the following structure: 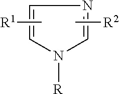
wherein each of R, R¹, and R² is independently a hydrogen atom or a methyl, ethyl, propyl, or butyl group, provided that R, R¹, and R² are not all hydrogen atoms;

19. The reagent of claim 14 wherein said molar ratio of said derivative of imidazole to said sulfur dioxide or derivative thereof is greater than 2:1.

20. The reagent of claim 14 that consists essentially of said sulfur dioxide or derivative thereof present in an amount of about 0.2 mols/liter of said reagent, said derivative of imidazole present in an amount of about 1.4 mols/liter of said reagent, and having the following structure: 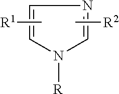
wherein each of R, R¹, and R² is independently a hydrogen atom or a methyl, ethyl, propyl, or butyl group, provided that R, R¹, and R² are not all hydrogen atoms;
said hydrogen halide donor present in an amount of about 0.3 mols/liter of said reagent and is a hydrohalide of a second derivative of imidazole wherein the second derivative of imidazole has the following structure: 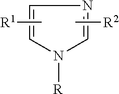
wherein each of R, R¹, and R² is independently a hydrogen atom or a methyl, ethyl, propyl, or butyl group, provided that R, R¹, and R² are not all hydrogen atoms; and
said solvent is an aprotic solvent and comprises a balance of the reagent and chosen from propylene carbonate, acetonitrile, N,N-dimethylformamide, N-methylformamide, acetamide formamide, N-methyl acetamide, N-dimethyl acetamide, 2-pyrrolidone, N-methyl-2-pyrrolidone, chloroform, dichloromethane, bromoform, dibromomethane, and combinations thereof.
